(19) United States
(12) Patent Application Publication
(10) Pub. No.: US 2017/0119374 A1
(43) Pub. Date: May 4, 2017

(54) CAM SUTURE RETENTION MECHANISM

(71) Applicant: Cook Incorporated, Bloomington, IN (US)

(72) Inventors: Brian L. Gaerke, Bloomington, IN (US); Thomas W. McGhie, Bloomington, IN (US)

(21) Appl. No.: 15/341,693

(22) Filed: Nov. 2, 2016

Related U.S. Application Data
(60) Provisional application No. 62/250,197, filed on Nov. 3, 2015.

(51) Int. Cl. A61B 17/04

(52) U.S. Cl. CPC A61B 17/0487; A61B 2017/0488

(57) ABSTRACT

There are disclosed embodiments for a suture retention mechanism. For example, embodiments may include a suture retention mechanism including a body with a suture aperture and a channel defined through the body. A cam is pivotally attached to the body so that the cam may be moved between an open position and a closed position. In the open position, a suture may move within the suture opening. In the closed position, the cam obstructs the suture opening so the suture may not move within the suture opening. An embodiment for a closure tool for a suture retention mechanism is also disclosed.

The present disclosure concerns devices for holding sutures used for various medical procedures. For example, it concerns an easy-to-use device for maintaining a suture that extends from the body in tension, with a minimal amount of discomfort for the patient. This application claims the benefit of U.S. Provisional Application No. 62/250,197, which is incorporated by reference herein in its entirety.

### BACKGROUND

Sutures are used in many medical procedures and for a number of purposes, such as repairing tissue, closing tissue openings and holding tissues or devices in particular locations or orientations. For example, in a gastropexy procedure, a suture anchor is used to hold a portion of the stomach against the abdomen wall as part of treatment for GERD (gastro-esophageal reflux disease), for insertion of a port or other access for the stomach, or for other purposes. A suture anchor may be placed through the skin and abdominal wall into the stomach, and used to move the stomach against the abdominal wall. The suture of the suture anchor extends through the skin, and is secured so as to maintain tension and hold the tissues in place.

It has been known in the past to tie off a suture end or to secure the suture to the skin, as by sewing through the skin. Suture retention mechanisms have been developed to hold such a suture, to maintain tension more securely and with less discomfort over time. In the specific example of a gastropexy procedure, as noted above a suture anchor in tension holds the stomach against the abdomen wall, and it may be necessary to retain such tension for a time period spanning multiple days or weeks. Therefore, it is desirable for the suture retention device to have the ability to be securely and easily locked to effectively hold the suture over time and to prevent the device from accidentally being removed from the patient. Because the device is on the outside of the skin in such cases, there is also a risk of irritation or injury to already-sensitive skin or other tissues, and of catching or snagging on exterior objects. Existing devices also include complicated locking mechanisms that can make the suture retention device difficult to install and lock. It has also been noted that existing clips or other devices can be turned, twisted or otherwise adversely moved by the tension of the suture or by the operation of the clip. Therefore, there is a need for improved suture retention devices.

### SUMMARY

Among other things, there is disclosed a suture retention mechanism. In particular embodiments, the suture retention mechanism includes a body or housing with a suture aperture for receiving a suture. The body includes a channel that is non-parallel (e.g. perpendicular) to the suture aperture through a side surface, which separates the body into an upper portion and a lower portion, and the suture aperture extends through both portions and the channel. The channel is at least partially bounded by one or more interior surfaces.

The suture retention mechanism also includes a cam lever with a forward camming surface, which in particular embodiments is a partially or entirely planar surface. The cam lever is positioned in and pivotally attached to the body within the channel, so that the cam lever is pivotable in the channel, e.g. between an open position and a closed position. The cam lever moves its camming surface within the channel, and the camming surface and/or another portion of the cam lever moves over or through the suture aperture while the cam is pivoted. Generally, the camming surface is adjacent to or faces at least a portion of the interior surface(s) within the channel.

The cam lever is pivotable within the channel around an axle, which in one embodiment is a pin. Such an axle may be fixedly attached to the body (e.g. the bottom and/or top portion) and through the cam lever so that the cam lever rotates around the axle, or may be fixedly attached to the cam lever and rotatable with respect to the body. In some embodiments, the cam lever may include a handle or handle portion. The handle may protrude from the body when the suture retention device is in an open position (e.g., a position in which the suture aperture is unobstructed or sufficiently so for a suture in the suture opening to be free to move within the suture opening). When the suture retention device is in a closed position (e.g., a position in which a part of the cam lever obstructs the suture aperture so that the suture cannot move within the suture opening), the cam lever and handle (if present) may be located entirely within the channel of the body. A particular embodiment has the outside edge of the cam lever or its handle flush with the outside edge of the body when the suture retention device is closed. In some embodiments, the cam lever may be locked in the closed position using detents or other locking mechanisms.

Further, embodiments are disclosed for suture retention mechanisms that include a cam lever that is pivoted so its camming surface contacts a suture inserted through the suture aperture when the cam lever is moved from the open position to the closed position. The camming surface pushes the suture to the side, so that the suture is crimped between the edge of the suture aperture and the cam lever, and/or so that the suture is pinched or gripped between the internal surface within the body's channel and the cam lever. In particular embodiments, the suture aperture of the suture retention mechanism includes an aperture edge. When the cam is in the closed position, the suture is held against the aperture edge. In the closed position, the cam may cover the suture aperture or the suture aperture may be open. In either case, the suture is prevented from moving within the suture aperture when the cam is in the closed position. The cam lever thus retains the suture via such crimping or pressure between the interior surface of the body and the cam lever so the suture is unable to move through the suture aperture, and tension in the suture is maintained.

In particular embodiments, a suture retention mechanism can include a disk-shaped body having an upper surface, a lower planar surface, and an external side surface, and a channel defined through the side surface and parallel with the lower planar surface separating the body into upper and lower portions, with the channel bounded in part by an upper face in the upper portion, a lower face in the lower portion, and one or more interior surfaces extending from the upper face to the lower face. The body further includes a suture aperture, the suture aperture having respective openings in the upper surface, upper face, lower face and lower surface to define a continuous straight path from the upper surface to the lower surface and through the channel. A cam lever is positioned in and pivotally attached by an axle to the body to allow the cam lever to pivot in the channel between an open position and a closed position. The exemplary cam lever can include a handle portion joined to an arm portion having an at least partially planar camming surface, so that the camming surface faces and moves along and past at least a portion of the interior surface of said body the cam is pivoted from the open position to the closed position. The axle may form an axis of rotation that is parallel to and offset from the suture aperture and is between the suture aperture and the external side surface, thereby intersecting the upper and lower faces. In such embodiments, when the cam lever is in the open position, the suture aperture is at least partially unobstructed to allow a suture located in the suture opening to move longitudinally within the suture opening, and when the cam is in the closed position, the cam obstructs the suture aperture so that a suture cannot move longitudinally within the suture opening.

Specific examples of such mechanisms may include a curved portion of the interior surface, with the suture aperture directly next to that curved portion; and/or a flared portion of the interior surface, with a portion of the handle facing that flared portion and an opposite portion of the handle is flush with or inside of the exterior side surface in the closed position. In the closed position a gap can exist between a tip of the handle and the body for access by a user to the handle. Particular examples of the handle include a length and opposing upper and lower surfaces and a tip, the tip having a diameter less than a width of the channel, and the distance between the upper and lower surfaces of the handle is not uniform along its length, so that at least one gap is formed between at least one of the upper and lower surfaces of the handle and at least one of the upper and lower faces of the body. The body may include at least one detent along the exterior side surface and extending from one of the upper and lower faces and into the channel. For example, first and second detents may be placed along the exterior side surface, each having a first height at the exterior side surface and respective portions sloping from the exterior side surface to one of the faces bounding the channel. Such detents may be directly opposite each other across the channel. The (or an) interior surface of the body may include a flat stop portion, so that a portion of the cam lever engages the flat stop portion when the cam lever is in a maximally-open position. As one example, when a portion of the cam lever engages the flat stop portion, an end portion of the arm of the cam lever contacts a curved portion of the interior surface.

In some embodiments, the axle may be a pin that extends through a portion of the cam lever and engages the body through openings in the upper and lower face of the body. The pin can be fixed with respect to one of the body and the cam lever. An exemplary body includes a pin aperture with respective openings in at least one of the upper surface and the lower surface of the body and in the upper and lower faces of the body for holding the pin. In other respects, a portion of the cam lever may protrude from the body when the cam is in the open position. When the cam lever is in said closed position, the cam lever could covers said suture opening, and an outside edge of the cam lever could be approximately flush with the exterior side surface of the body. The opening in the lower surface of the body of the suture aperture is in the center of the lower surface of the body in a particular example.

Other particular embodiments of a suture retention mechanism include a body having a suture aperture, the suture aperture having a longitudinal axis and respective openings in an upper surface and a lower surface to define a continuous straight path from the upper surface to the lower surface. A cam lever is positioned in and pivotally attached by an axle to the body to allow the cam lever to pivot into and out of the body between an open position and a closed position, the cam lever including an arm portion with a side camming surface. The axle forms an axis of rotation that is parallel to and offset from the longitudinal axis of the suture aperture and is between the suture aperture and an external side surface of the body, and when the cam lever is pivoted around the axis of rotation toward the closed position the camming surface moves laterally across the suture aperture. Examples of the body can include a channel into and out of which the cam lever is pivotable, the channel being partially bounded by a plurality of interconnected internal surfaces. One of the internal surfaces is a stop surface that engages the cam lever at a maximally-open position to prevent further outward pivoting of the cam lever, one is a curved surface adjacent the suture aperture and facing the camming surface of the cam lever, and one is a flared surface defining an area for accommodating a handle portion of the cam lever when the cam lever is in the closed position. The body may include at least one detent for holding the cam lever in the closed position. The cam lever may include a curved handle portion that extends outside of the external side surface of the body when the cam lever is in the open position, and is flush with or inside of the external side surface of the body when the cam lever is in the closed position. For instance, when the cam lever is in the closed position, at least one gap exists between the handle portion and the body to assist a user in accessing the handle portion to open the cam lever. The cam lever may include a ridge that snaps over a projection within the body as the cam lever goes to or is moved to the closed position.

Also disclosed is a closure mechanism or tool usable to close a suture locking mechanism such as those described herein. Embodiments of such a closure mechanism can include a base having an inner surface for engaging a first part of the suture locking mechanism, a concave surface at a first end and a slot at a second end. The slot may be defined at least in part by upper and lower surfaces, each having a respective groove, with each respective groove having a curved entry portion and a rounded end portion. A bearing member has an inner surface for engaging a second part of the suture locking mechanism, the bearing member having a concave surface at a first end and a tongue at a second end. The tongue includes an upper and lower surface, each of which has a respective projections having convex end surfaces, one convex side surface and one concave side surface. The respective projections are located within and pivotable around a pivot axis within the respective rounded end portions of the respective grooves. When the closure mechanism engages the suture locking mechanism to close the suture locking mechanism the pivot axis is aligned with an axis about which the second part of the suture locking mechanism rotates with respect to the first part. In particular embodiments, when the suture locking mechanism is closed sufficiently to lock a suture, the respective projections are slidable through the entry portion of the respective grooves so that the base and bearing member can be disassembled. The base and bearing member may be pre-assembled and engaged to the suture locking mechanism prior to receipt and use by a user.

### BRIEF DESCRIPTION OF THE DRAWINGS

FIG. 1 is a perspective view of an embodiment of a suture retention mechanism.

FIG. 2 is a top view of the body of the embodiment shown in FIG. 1, without the suture shown in FIG. 1. The bottom view is a mirror image.

FIG. 3 is a cross-sectional view of the embodiment shown in FIG. 2 in an open position.

FIG. 4 is a perspective view of a portion of the embodiment shown in FIG. 2.

FIG. 5 is a perspective view of the embodiment shown in FIG. 4.

FIG. 6 is a perspective view of a portion of the embodiment of FIG. 2.

FIG. 7 is a perspective view of the cross-section shown in FIG. 3, and showing a suture.

FIG. 8 is a top view of the cross-section shown in FIG. 7, and in a closed position.

FIG. 9 is a perspective view of a portion of an embodiment of a suture retention mechanism.

FIG. 10 is a perspective view of the portion of the embodiment shown in FIG. 9.

FIG. 11 is a side view of the portion of the embodiment shown in FIG. 10.

FIG. 12 is a cross-sectional view of the portion of the embodiment shown in FIG. 10.

FIG. 13 is a perspective view of a portion of an embodiment of a suture retention mechanism usable with the portion shown in FIG. 9.

FIG. 14 is a perspective view of the portion of the embodiment shown in FIG. 13.

FIG. 15 is a top view of the portion of the embodiment shown in FIG. 13. The bottom view is a mirror image.

FIG. 16 is a part cross-sectional view of an embodiment of a suture retention mechanism combining the portions shown in FIGS. 9 and 13 in a closed position.

FIG. 17 is a part cross-sectional view of the embodiment shown in FIG. 16 in an open position.

FIG. 18 is a top view of a closure tool usable with embodiments of suture retention mechanisms as described herein.

FIG. 19 is a perspective view of a portion of the embodiment shown in FIG. 18.

FIG. 20 is a perspective view of the portion of the embodiment shown in FIG. 19.

FIG. 21 is a perspective view of the portion of the embodiment shown in FIG. 19.

FIG. 22 is a perspective view of a portion of the embodiment shown in FIG. 18.

FIG. 23 is a perspective view of the portion of the embodiment shown in FIG. 22.

FIG. 24 is a perspective view of the portion of the embodiment shown in FIG. 22.

FIG. 25 is a top view of the combination of the embodiment shown in FIG. 18 and the embodiment shown in FIG. 16, in an initial or open position.

FIG. 26 is a top view of the combination shown in FIG. 25, in a closed position.

### DETAILED DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated embodiments, and such further applications of the principles of the disclosure as illustrated therein being contemplated as would normally occur to one skilled in the art to which the disclosure relates.

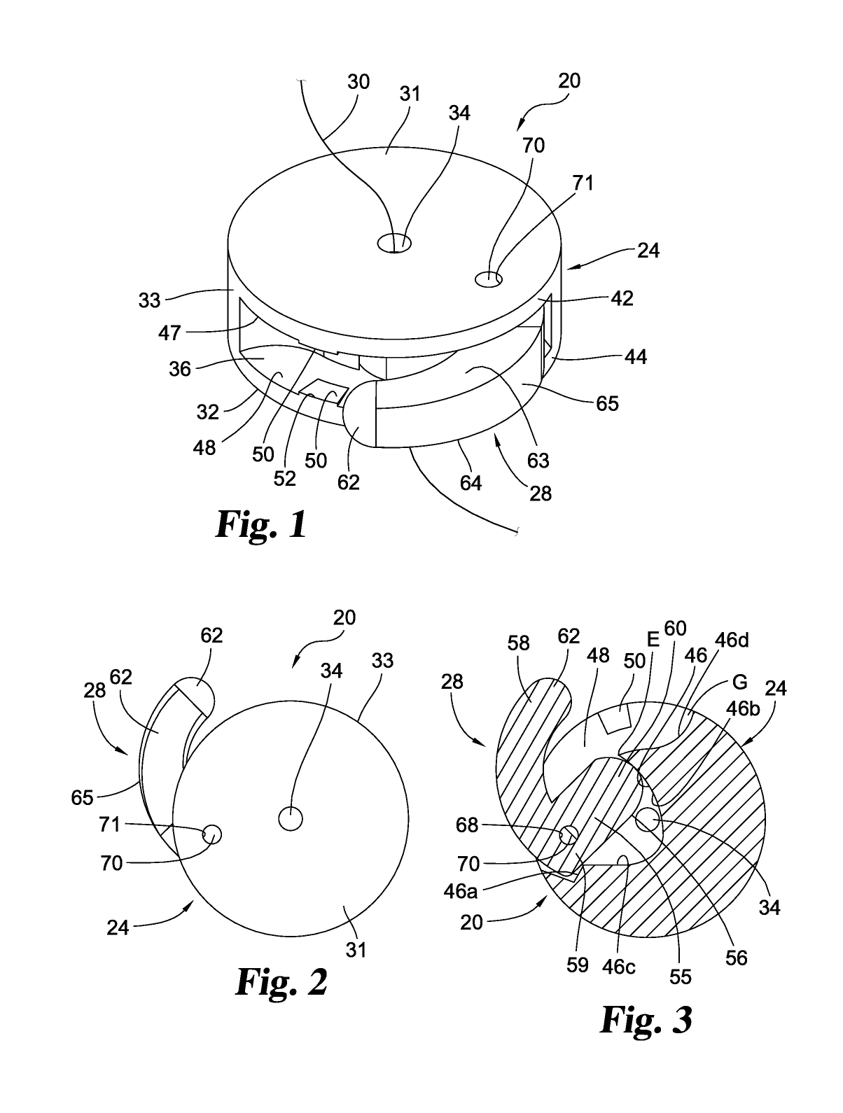

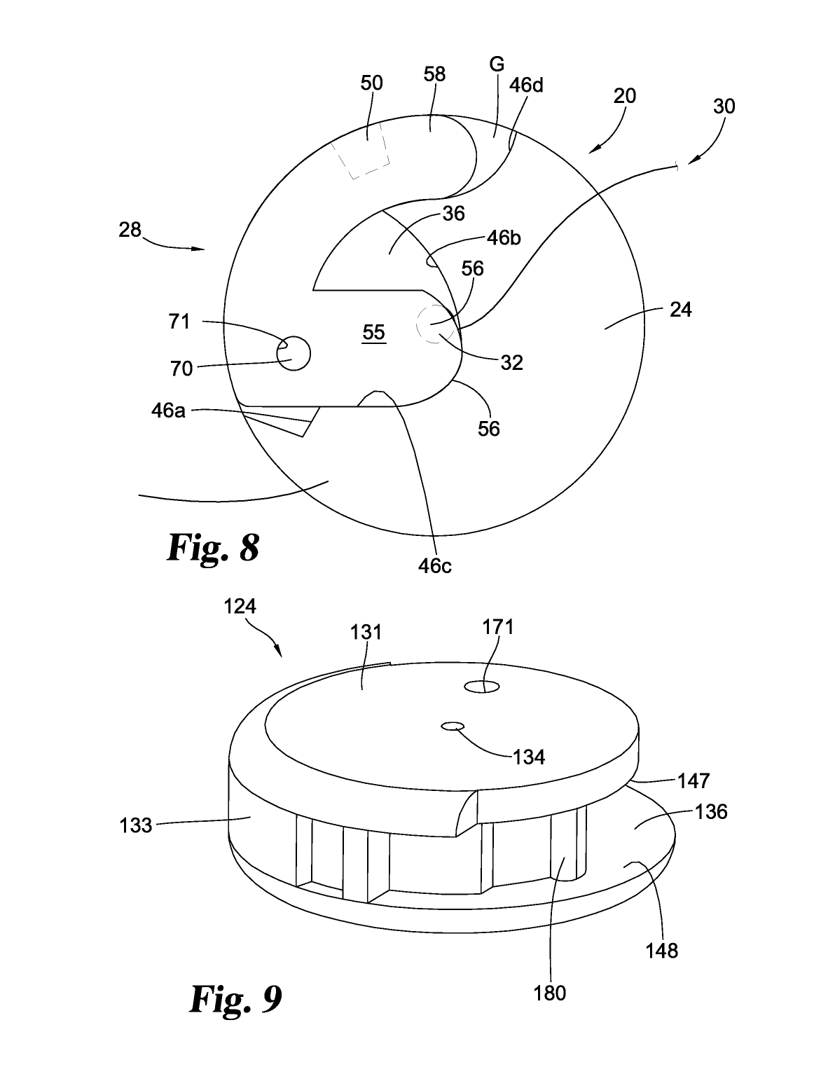

Referring now generally to the drawings, embodiments of a suture retention mechanism 20 are shown. FIG. 1 shows a view of an embodiment of suture retention mechanism 20 that includes a housing or body 24 and a cam lever 28. A suture 30 passes through body 24 and to the side of cam lever 28. As will be explained further below, by moving cam lever 28 from an open position (e.g. FIG. 1) to a closed position (e.g. FIG. 8), the suture passing through body 24 is crimped, gripped or otherwise held in tension. When it is desired to remove mechanism 20, cam lever 28 is opened and mechanism 20 is removed from the suture.

Body 24 in the illustrated embodiment is a cylindrical or disc-shaped body, having an upper surface 31, a lower surface 32 and a side surface 33, and preferably has a small height to present a minimal profile when in use against a patient's skin. Rounded edges are preferred so as to reduce the discomfort the patient experiences. For example, a planar lower surface 32 and/or upper surface 31 may be provided, with the edges between such surfaces and side surface 33 rounded, e.g. having a part-toroidal shape. As another example, one or both of surfaces 31 and 32 may have a convex, conical or other tapering or sloping configuration, e.g. where body 24 is thickest in or near the center and thins as side surface 33 or a rounded edge is approached. It will be seen that a number of other shapes (e.g. a rectangular solid) could be used for body 24 consistent with the present disclosure, although a cylinder or disc is believed to be most efficient and effective. Materials for body 24 can include any sturdy material, and preferably would feature materials that are not irritating to the patient's skin (e.g. sturdy plastics such as acrylonitrile butadiene styrene (ABS) or other hypoallergenic substances).

Body 24 includes a suture aperture 34, which passes through top surface 31 and bottom surface 32, and is sized to allow a suture to pass through. In the illustrated embodiment, suture aperture 34 is linear, i.e. straight or uncurved, and is centrally-located (e.g. at the center) and perpendicular to surfaces 31 and 32 and parallel to the curved side surface 33. Body 24 also includes a groove or channel 36 between surfaces 31 and 32, which in the illustrated embodiment is parallel to and equidistant from surfaces 31 and 32. As shown in FIG. 2, channel 36 separates a top portion 42 and a bottom portion 44 of body 24. Channel 36 is sized to allow at least part of cam lever 28 to fit within it, and in an illustrated embodiment, channel 36 is large enough to allow the entirety of cam lever 28 to fit inside it. For example, in such a closed condition cam lever 28 may form a part of or be coextensive with side surface 33 of body 24. In other embodiments, only a portion of cam lever 28 may fit within channel 36, e.g. to allow a part to extend or reach outside of side surface 33, for instance to ease outward pivoting of cam lever 28 for release and/or adjustment of retention mechanism 20 with respect to suture 30.

Channel 36 in the illustrated embodiment extends inward from side surface 33 of body 24 to one or more interior surface(s) 46 that extend the width of channel 36, between top portion 42 and bottom portion 44, and is bounded by two parallel surfaces 47, 48 facing channel 36. Interior surface(s) 46 are perpendicular to faces 47, 48 and parallel to the axis of suture aperture 34 in this embodiment. Surface(s) 46 can include a planar portion (46a) that functions as a stop or boss for cam lever 28, preventing it from being opened too far; a curved or cylindrical portion (46b) that guides or mates with a curved portion of cam lever 28; a planar portion (46c) extending from portion 46b; and a curved or flared portion 46d forming an edge E with portion 46b, for accommodating cam lever 28 when closed, and providing a gap G between cam lever 28 and portion 46d to enable opening of device 20 if desired. It will be understood that the shape or number of surface(s) 46 may vary depending on the shape or size of cam lever 28 in different embodiments. Channel 36 and interior surfaces 46 are dimensioned in this embodiment to allow the entirety of cam lever 28 to fit within body 24 when device 20 is closed. Some embodiments may include one or more detents 50 within channel 36 to help secure cam lever 28 in a closed position, e.g. via a press, interference or snap fit. The illustrated embodiment of detents 50 is a trapezoidal ramp having one end that merges into one of the surfaces 47, 48, and an opposite end forming a tooth or boss 52.

Cam lever 28 in the illustrated embodiment includes an engagement portion or arm 55 with a camming surface 56 (see FIG. 3) and a handle portion 58. Arm 55 and handle 58 may be formed monolithically. Arm 55 is generally linear in this embodiment, having a first end 59 connected to handle portion 58 and a second end 60 that includes at least part of camming surface 56. Arm 55 is also of a uniform thickness, the same as or slightly smaller than the width of channel 36. Camming surface 56 is planar (56a) along a side of arm 55 and curves (56b) toward end 60 of arm 55 in this embodiment. Alternatively the forward portion 56b could also be planar or have a planar part, in the same plane as or forming an obtuse angle with portion 56a, while in other embodiments camming surface 56 may have another shape that allows rotation of cam lever 28. End 60 has a configuration compatible with surface 46b of body 24 in an illustrated embodiment, so that as cam lever 28 is rotated within body 24 surface 60 is guided by or passes along most or all of surface 46b. Camming surface 56 generally faces one or more portions of interior surface 46. For example, when device 20 is open, camming surface 56 may face one or both of surfaces 46c and 46b, while when closed camming surface 56 faces surface 46b and/or 46c in the illustrated embodiment.

Handle portion 58 extends from end 59 of arm 55, to allow a physician or other user to easily pivot cam 28 with respect to body 24. In the illustrated embodiment handle 58 curves generally toward arm 55 in a curve that is the same as the curve of side surface 33 of body 24. In other embodiments, handle portion 58 may have a different curvature or be straight, so that handle 58 may be pushed, pulled or otherwise manipulated to allow the user to pivot cam lever 28. Handle 58 has a generally rounded (e.g. part-spherical) tip 62, and upper, lower and side surfaces 63, 64, 65 (respectively) extend from tip 62 toward arm 55. In the illustrated embodiment, tip 62 has a maximum diameter or thickness that is less than the width of arm 55 and channel 36. Upper surface 63 and/or lower surface 64 meet tip 62 and extend to meet arm 55 at smooth junctions. Accordingly, in the illustrated embodiment at least one of surfaces 63, 64 are sloped relative to arm 55 and channel 36, or the width of handle 58 measured between surfaces 63 and 64 is not uniform, but is larger at a point adjacent arm 55 than at a point adjacent tip 62. A space thus exists between (a) tip 42 or one or both surfaces 63, 64 and (b) a facing surface 47 and/or 48 when cam lever 28 is in the closed position. It will also be seen that the configuration of tip 62 permits a space to exist between tip 62 and surface 46d and/or side surface 33. Such space(s) permits easier access to handle 58 when a user desires to remove or adjust mechanism 20.

Arm 55 includes a cylindrical opening or shaft 68 in this embodiment, through which an axle 70 (shown as a pin in one embodiment) extends, so that the axis of rotation of cam lever 28 passes through arm 55. Axle 70 is of stainless steel in one example, and forms a stable pivoting connection between cam lever 28 and body 24. Axle 70 may be fixed with respect to body 24 in openings 71 so that cam lever 28 rotates around axle 70, or axle 70 may be held by but pivotable with respect to body 24 in openings 71. In other embodiments, axle 70 may be a part of arm 55, for example detents or projections (not shown) that snap-fit into openings (e.g. openings 71) in body 24. In the illustrated embodiments, axle 70 is not exposed to the side, but is entirely inside the perimeter of side surface 33, and is parallel to suture aperture 34. Such placement of axle 70 provides for stable operation of mechanism 20, because there is less or no tendency for mechanism 20 to turn around the axis of suture 30 or suture aperture 34, and so no inclination to twist suture 30. The illustrated example also shows axle 70 separated from aperture 34, e.g. so that the distance between axle 70 and aperture 34 is between three-fourths and two-thirds of the radius of body 24. That exemplary location provides a satisfactory combination of stability with mechanical advantage in movement of end 60 of arm 55 of cam lever 28.

FIG. 3 shows a cross-section of suture retention mechanism 20 with cam lever 28 in an open position relative to body 24. In the open position, cam lever 28 is oriented so that arm 55 is clear of suture opening 34, leaving suture opening 34 wholly or partially unobstructed sufficiently to allow a suture to move longitudinally through opening 34. In this position, a suture 30 may be passed or moved through the entire distance of suture opening 34 from top surface 31 to bottom surface 32. In the illustrated embodiment, handle portion 58 of cam lever 28 extends outside of channel 36 and the outer circumference of suture retention mechanism 20 (side surface 33) when cam lever 28 is in an open position.

FIG. 8 shows a cross-section of suture retention mechanism 20 with cam lever 28 in a closed position. In the closed position, cam lever 28 is oriented so that arm 55 covers suture opening 34 sufficiently to crimp, grip or pinch a suture through opening 34. From the open position, as cam lever 28 is pivoted so that handle 58 moves toward side surface 33 of body 24 and arm 55 moves toward surface(s) 46, arm 55 moves over suture opening 34, led by camming surface 56. Camming surface 56 slides along or in front of a portion of surface 46b. Suture 30 within suture aperture 34 is contacted and pushed by camming surface 56 as cam lever 28 is pivoted. Further pivoting pushes suture 30 to the edge of aperture 34 and then to be crimped between arm 55 and an edge of suture opening 34, and/or gripped or pinched between camming surface 56 and interior surface 46 (see FIG. 7), so that suture 30 cannot move longitudinally through suture aperture 34.

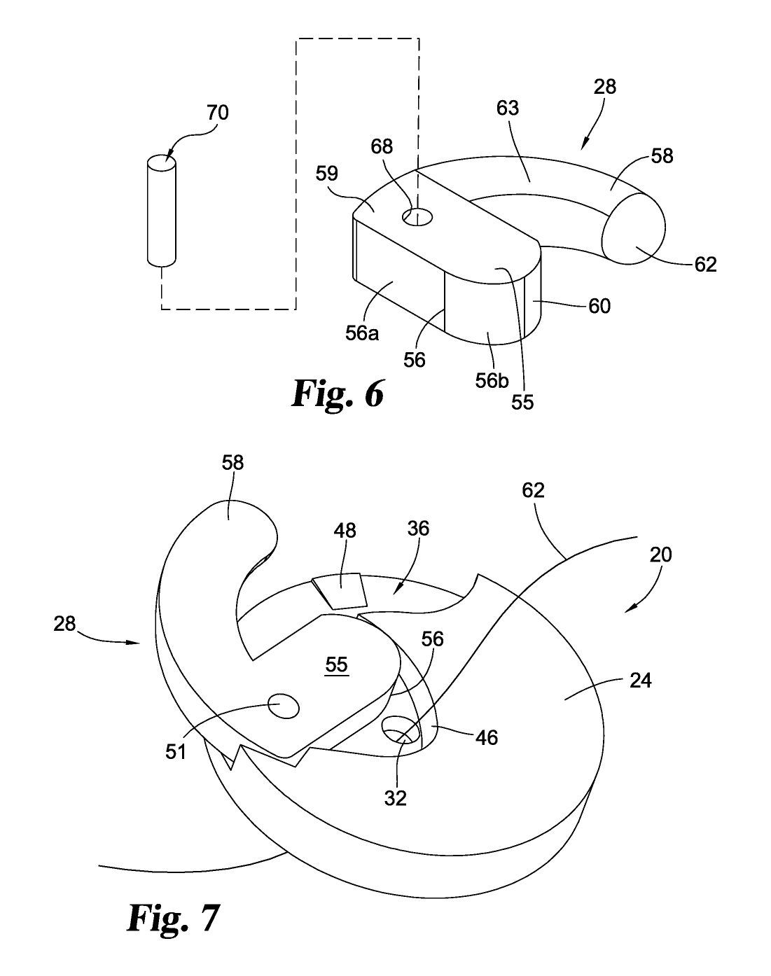

Cam lever 28 obstructs suture aperture 34 in the closed position so as to prevent suture 30 from moving within suture aperture 34. As shown in FIG. 6, this may be accomplished when cam portion 55 covers suture aperture 34. However, in other embodiments, cam lever 28 may hold suture 30 without actually covering or being immediately above all of suture aperture 34. Instead, cam lever 28 may only retain suture 30 between camming surface 56 and interior surface(s) 46 (e.g. surface 46b and/or 46c) so suture 30 cannot move in suture aperture 34, without covering some of suture aperture 34. That is, embodiments may not require the suture to be twisted along its axis or assume the form of a portion of cam lever 28 as it travels through aperture 34.

In the embodiment shown in FIG. 6, handle 58 of cam lever 28 is located entirely within channel 36 when cam 28 is in a closed position. This gives suture retention mechanism 20 a smaller profile, making anchor 20 more comfortable for a patient and decreases the risk of suture retention mechanism 20 getting caught on clothing or other materials. In other embodiments, however, a portion or the entirety of handle 58 may protrude from body 24 when cam 28 is arranged in a closed position.

In some embodiments, cam lever 28 may be biased or locked into the closed position, so suture retention mechanism 20 does not become accidently unlocked once installed. As one example, cam lever 28 may be locked into the closed position is using detents 50. One or more detents 50 protrude from the top portion 42 of body and/or bottom portion 40 of body 24; the illustrated embodiment shows one detent 50 extending from both of surfaces 47 and 48, directly opposed to each other. Detent(s) 50 keep cam lever 28 secured in the closed position, but allow cam lever 28 to be pivoted to the open position by applying force on handle 58 sufficient to overcome the interference between the associated detent(s) and depression(s). Other methods of locking such as a spring bias or a mechanical lock may also be used.

In use, if not already in an open position the user pivots cam lever 28 of mechanism 20 within channel 36 so cam lever 28 is in an open position. To pivot cam 28 into an open position, handle 58 is pulled outward from body 24 so that force applied to cam 28 is applied in a substantially lateral direction relative to body 24 rather than downward or upward. When suture retention mechanism 20 is used on a patient, it is generally placed in an area where the patient will already be experiencing pain. Applying an upward force on a part of mechanism 20 can introduce slack in the suture if mechanism 20 is not fixed to the suture, and pulls on the suture which is already in high tension if mechanism 20 is fixed to the suture. Applying a downward force to mechanism 20 can also introduce slack as well as giving pressure to the already painful area on the patient. By operating cam 28 with lateral force, the amount of pressure directed onto the patient and along the length of the suture is minimized or eliminated and pain and discomfort is reduced.

If not pre-loaded, mechanism 20 is then placed onto suture 30 by threading suture 30 through suture aperture 34 in suture retention mechanism 20. Mechanism 20 is moved along suture 30 to a desired location, e.g. against the patient's skin or a bandage on the skin, with the desired tension maintained on suture 30. Once mechanism 20 is in the desired location, cam lever 28 is pivoted into a closed position, for example, by pushing handle 58 toward body 24 and into channel 36. Pushing handle 58 pivots cam lever 28 around axle 70, so that camming surface 56 pivots within channel 36 with respect to body 24 and slides along interior surface 46 of body 24. As camming surface 56 slides along interior surface 46, it comes into contact with the portion of suture 30 within channel 36. As cam lever 28 continues to pivot, camming surface 56 pushes suture 30 across aperture 34, and suture 34 slides or moves along surface 56. Suture 30 is bent, crimped, pinched or gripped over an edge of aperture 34 and/or between surface 56 and surface 46 as cam lever 28 arrives at the closed position.

As will be understood, the present mechanism 20 does not rely on wrapping the suture around a cylinder or pivoting portion of a device. Suture 30 remains to the side of camming surface 56 and the rest of cam lever 28, and does not go through or wrap around any part of cam lever 28. Suture 30 moves laterally to its own axis and with respect to axle 70, rather than around the axis of rotation of cam lever 28 (e.g. axle 70).

By crimping or gripping suture 30 between the camming surface 56 and interior surface 46, the suture can no longer move within suture aperture 34, and the position of mechanism 20 along suture 30 cannot be changed. Therefore, mechanism 20 holds suture 30 in a desired location and in a desired tension until cam lever 28 is out of the closed position, as for adjusting or removal. The pivoting cam structure allows the suture anchor to be easily locked and unlocked using only one hand. A user's other hand is free to perform other tasks as may be needed during the gastropexy or other procedure.

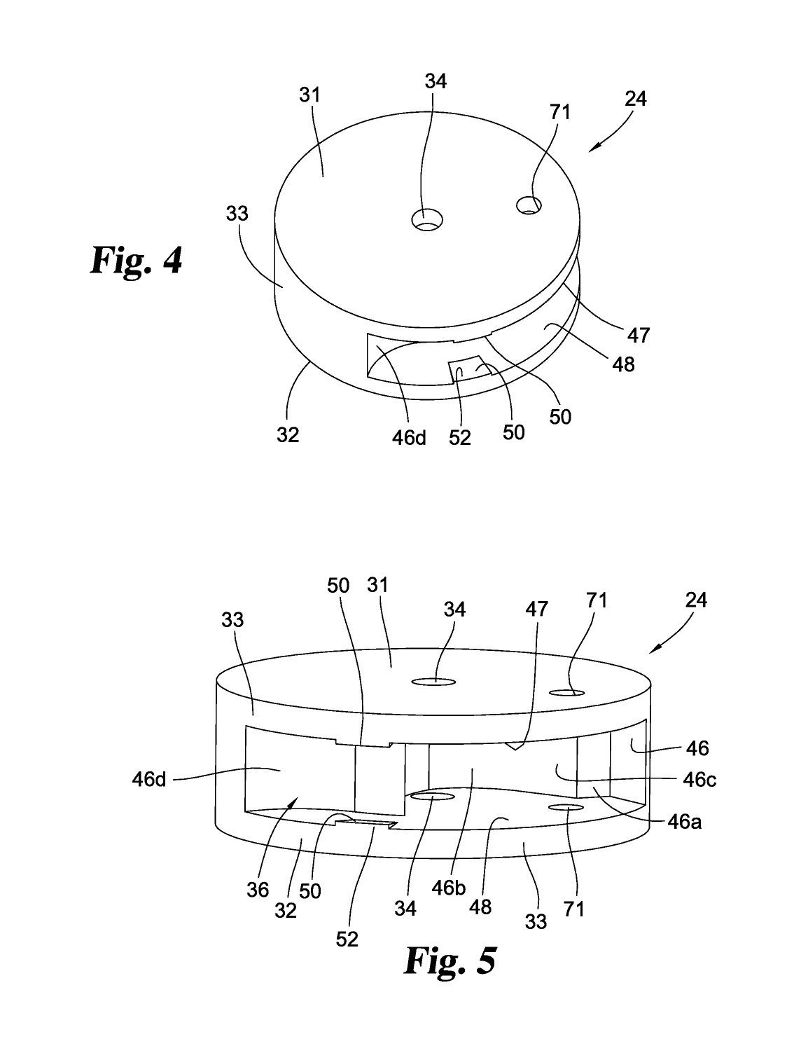

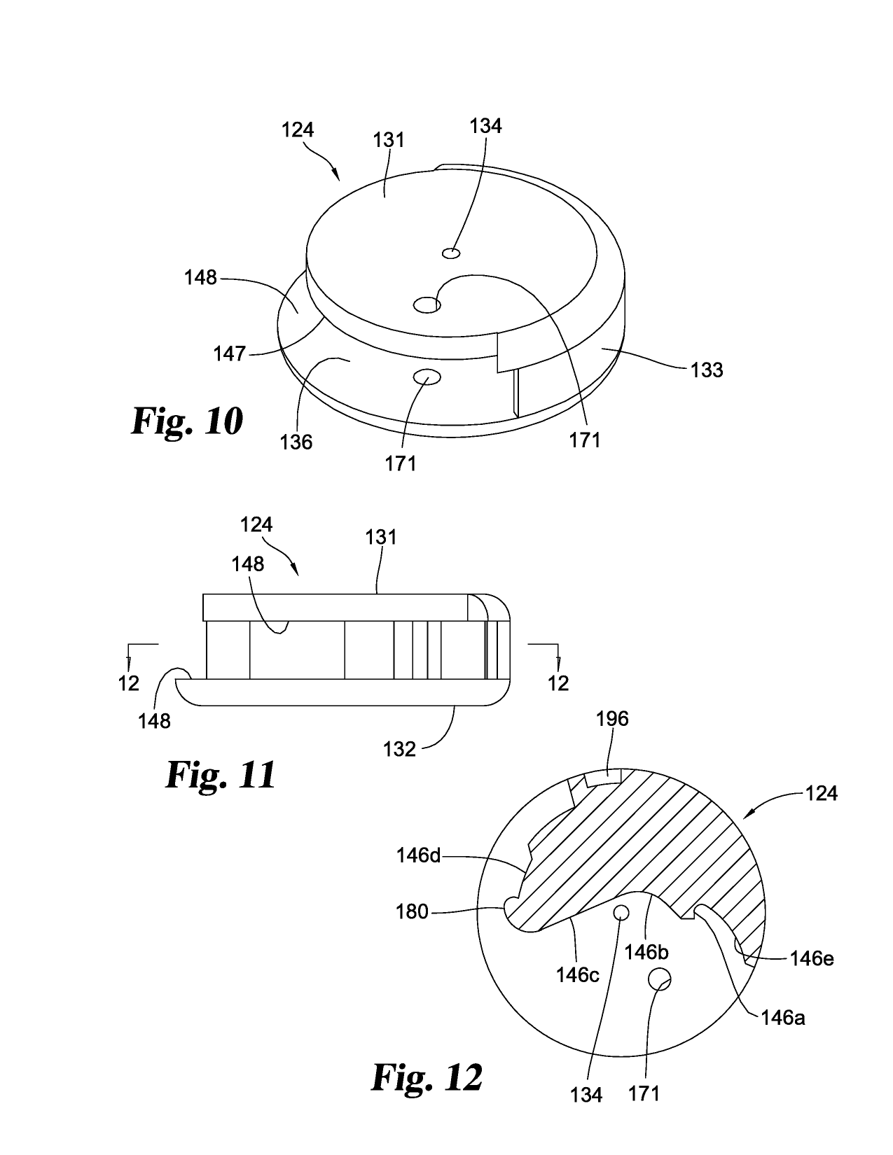

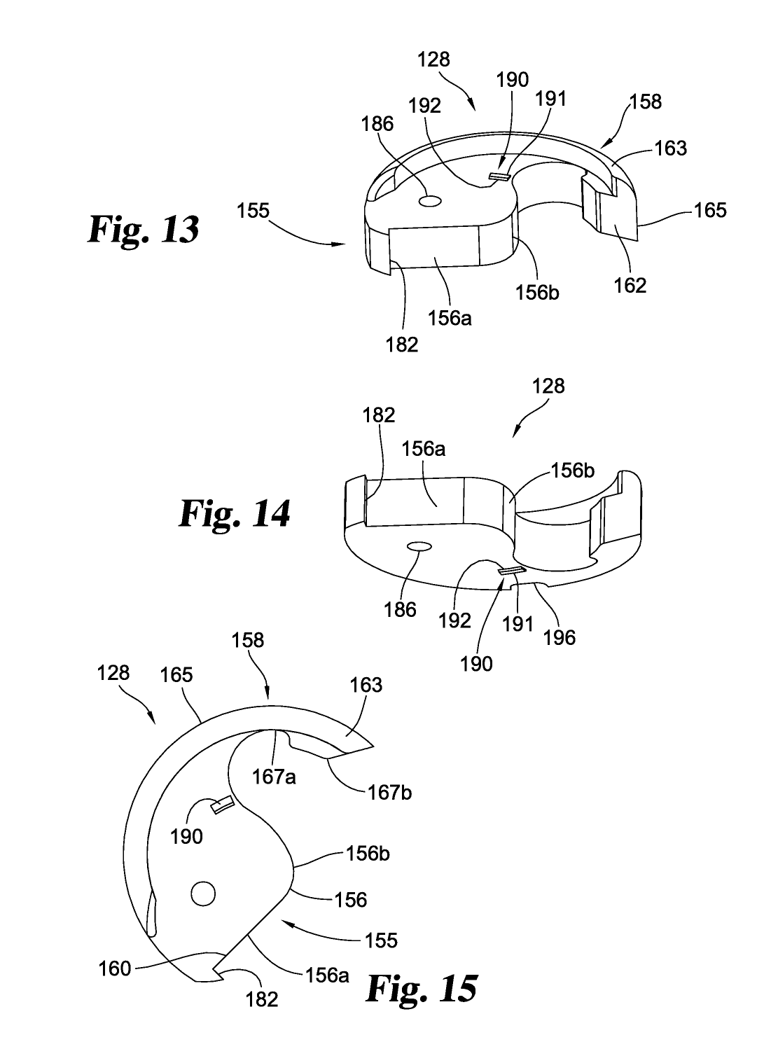

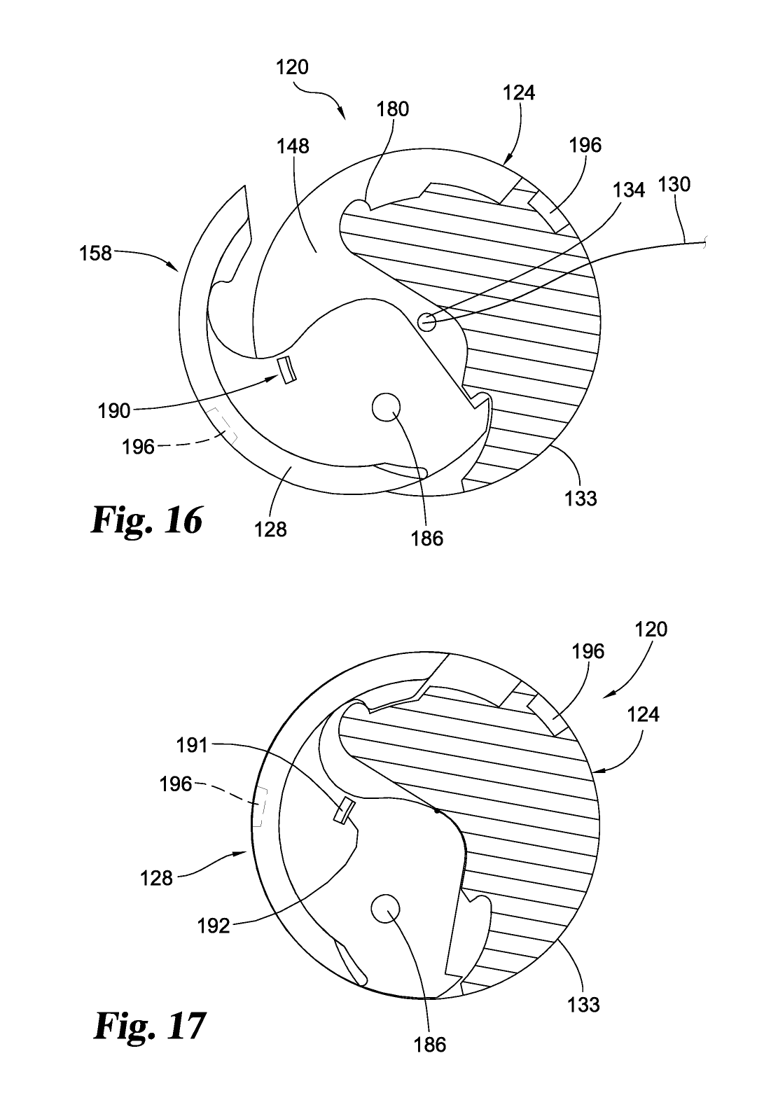

Another embodiment of a suture retention mechanism 120 is shown in FIGS. 9-17, in which similar or identical parts have the same numbers used in FIGS. 1-8 with the addition of the prefix 1. Mechanism 120 includes a body 124 and a cam lever 128 interengaged via an axle 170, so that cam lever 128 can move between an open position and a closed position in which a suture 130 passing through body 124 is crimped, gripped, pinched or otherwise held within mechanism 120. Opening cam lever 128 permits adjustment or removal of mechanism 120 from suture 130.

Body 124 is configured generally the same as described above, with an upper surface 131, a lower surface 132 and a side surface 133, with the connections between surfaces 131 and 133 and between 132 and 133 being rounded. Lower surface 132 is a circular area in this embodiment, while upper surface 131 does not extend laterally to the same extent as lower surface, but has a first circular edge portion that is at the same radius as the edge of lower surface 132, and a second circular edge portion concentric with that first portion but with a smaller radius. Suture aperture 134 is linear and oriented centrally and perpendicularly to surfaces 131 and 132 in this embodiment. Groove or channel 136 extends parallel to surfaces 131 and 132 and provides space for cam lever 128 to move within body 124 and across suture aperture 134. As will be seen, in this embodiment a portion of cam lever 128 remains outside of channel 136 when in the closed position.

Channel 136 in this embodiment extends inward, similar to channel 36 described above, with interior surface(s) 146 and upper and lower surfaces 147, 148. Surfaces 146 in this embodiment include a stop portion 146a which may be planar; a portion 146b that guides or mates with a portion of cam lever 28, and in this embodiment is partially curved or cylindrical (146b) and partially planar (146c); and a curved or flared portion 146d for accommodating and locking cam lever 28 when closed. A further section 146e which is curved so as to guide a portion of cam lever 128 as that portion moves toward or away from surface 146a. Surface(s) 146 of body 124 in this embodiment includes a bulbous projection 180, which in the illustrated embodiment is a part of one or both of portions 146c and 146d, or may be considered a separate portion between portions 146c and 146d. As will be described further below, projection 180 forms a part of a snap connection with a rounded opening or depression in cam lever 128.

The illustrated embodiment of cam lever 128 is quite similar to cam lever 28, having an engagement portion or arm 155 with a camming surface 156 and a handle portion 158. Arm 155 is a generally planar body in this embodiment, of a uniform thickness the same as or slightly smaller than the width of channel 136, and with an end 160 generally opposite handle portion 158 that includes at least part of camming surface 156 and a boss or stop surface 182. Camming surface 156 is planar (156a) along a side of arm 155 adjacent boss 182 and curves (156b) toward handle 158. The configuration of camming surface 156 in this embodiment forms a close or snug fit of at least part of portions 156a and 156b with an opposing portion of surfaces 146b and 146c of body 124 when mechanism 120 is in the closed position.

Handle portion 158 curves generally toward arm 155 in a curve that is the same as the curve of side surface 133 of body 124. Handle 158 has a generally planar tip 162 in this embodiment. A rounded upper surface 163 extends from tip 162 along the outside of handle 158 and at least a portion of the outside of arm 155 toward boss 182, and in the closed position of device 120 forms with an edge portion of body 124 a generally continuous rounded upper edge of device 120. A side surface 165 extends along cam lever 128, through handle portion 158 and arm portion 155, and in this embodiment is continuous with side wall 133 of body 124 when device 120 is in the closed position. On the inside of handle 158 is a snap channel 167a bordered by a tooth or ridge 167b. Channel 167a and ridge 167b are configured to be able to be forced over bulbous projection 180 of body 124 so that projection 180 is within channel 167a and is held in that position in a snap fit.

Arm 155 may include an opening or shaft for an axle, as described further above with respect to arm 55. The illustrated embodiment has instead a pair of opposed projections 186 (shown as portions of a sphere, e.g. a hemisphere, in one example) extending from opposite sides of arm 155 and perpendicular to upper and lower surfaces of arm 155. Projections 186 are compatible with openings or shaft 171 in body 124, so that cam lever 128 is pivotally connected to body 124 and the axis of rotation of cam lever 128 with respect to body 124 passes through arm 155.

Cam lever 128 includes one or more bosses 190 for holding cam lever 128 in an open position (i.e. to keep it from prematurely closing) prior to or during use. Two such bosses 190 are shown in this embodiment, extending opposite each other at a location inside the rounded upper edge of cam lever 128. Bosses 190 may be placed on handle portion 158, on arm 155, or bridging them. Bosses 190 have an upper surface 191 that is parallel to the upper and lower surfaces of arm 155, and to the facing surfaces 147, 148 of body 124, and an inward-facing surface 192 that is oblique (e.g. about 45 degrees) with respect to the surface of cam lever 128 to which it is attached.

This embodiment of device 120 includes at least one side aperture 196, which may be used to hold device 120 or a part of it during placement and/or closing of device 120. Aperture 196 may be rounded or substantially rectangular, and the illustrated embodiment includes at least one aperture 196 in body 124 (e.g. in side surface 133) and at least one aperture 196 in cam lever 128 (e.g. in side surface 165). Additional side apertures (not shown) may be positioned adjacent aperture(s) 196 (e.g. above, below or to the side) or in other positions (e.g. around side surfaces of body 124 and cam lever 128).

Operation of mechanism 120 is essentially the same as the operation of mechanism 20 discussed above. Once mechanism 120 is loaded on a suture and in the desired location with respect to the patient, cam lever 128 is pivoted into a closed position, for example, by pushing handle 158 toward body 124 and into channel 136. Pushing handle 158 pivots cam lever 128 around the axle formed by projections 186 within openings 170, so that camming surface 156 pivots within channel 136 with respect to body 124. As cam lever 128 continues to pivot, camming surface 156 pushes suture 130 across aperture 134, and suture 30 is bent, crimped, pinched or gripped over an edge of aperture 134 and/or between surface 156 and surface 146 as cam lever 128 arrives at the closed position. Handle 158 is forced into body 124 until ridge 167b is forced over projection 180, snapping projection 180 into channel 167a. Mechanism 120 can be reopened, if necessary or desired, by unsnapping cam lever 128, e.g. by pulling on handle 158 until ridge 167b is forced back over projection 180.

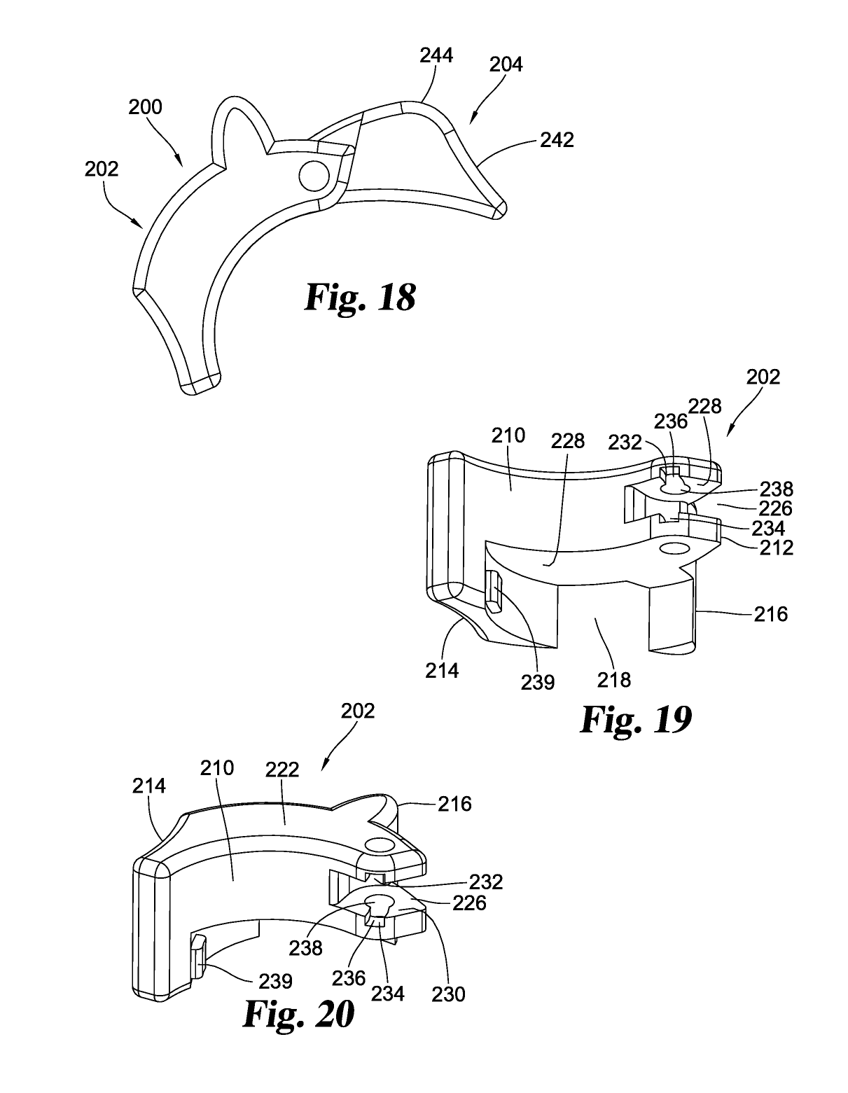

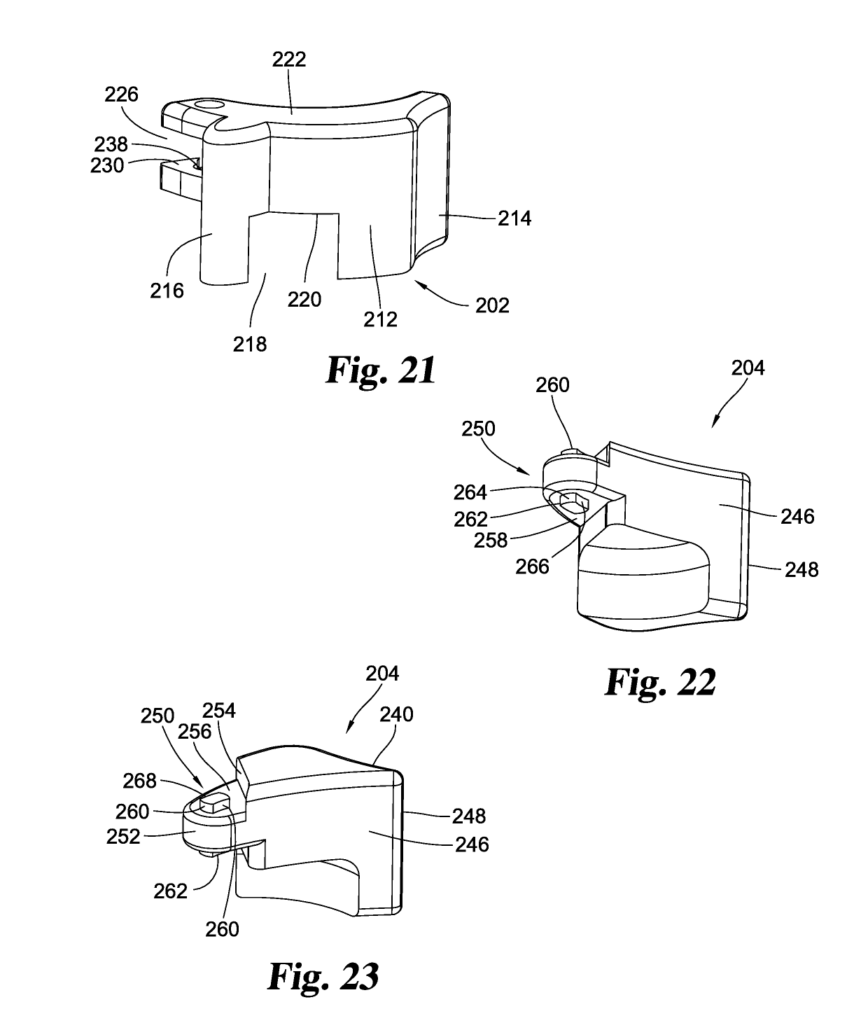

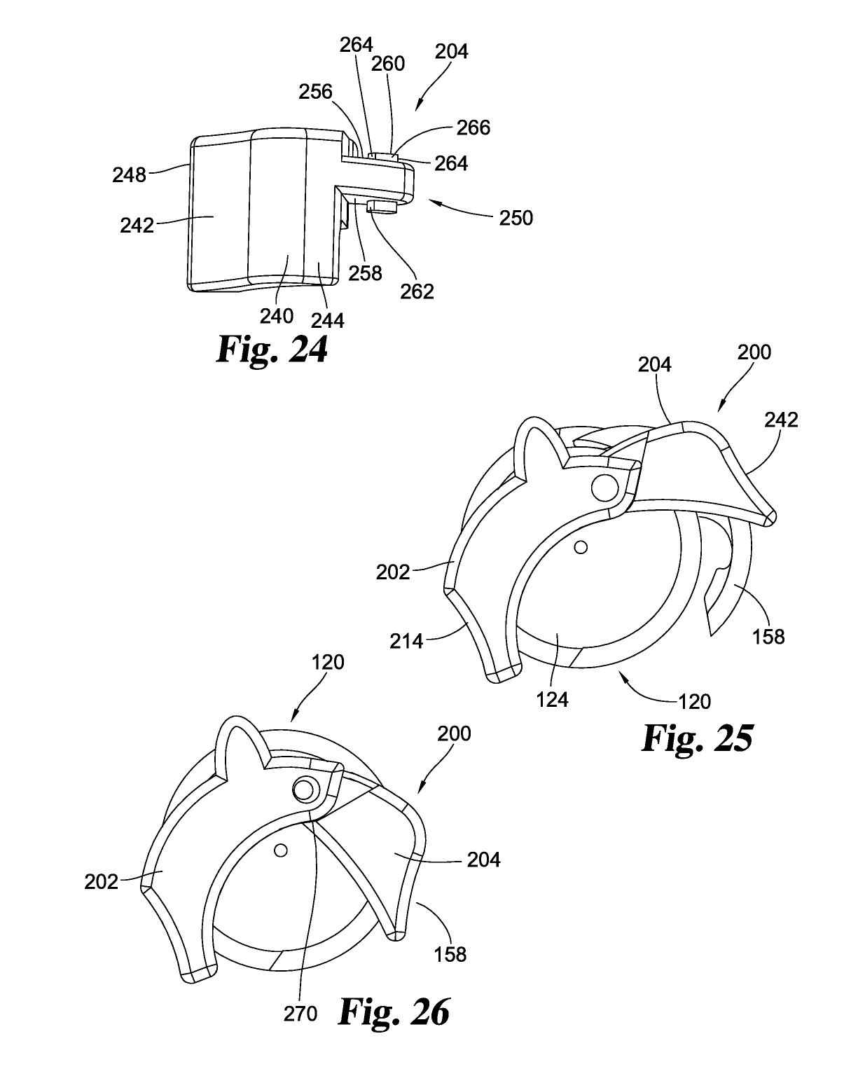

FIGS. 18-26 show an embodiment of a closing tool 200 that can be used to move device 120 or another suture locking mechanism from an open position to a closed position. Tool 200 includes a base 202 and a bearing member 204 pivotally connected to base 202. In particular usages, base 202 acts as a brace or block to be placed against a suture locking mechanism and held substantially fixed by the user (e.g. the user's thumb or finger, or a tool held by the user). Bearing member 204 engages another part of a suture locking mechanism and is pushed by the user, e.g. by a finger or tool, pivoting around a pivot axis toward base 202 and closing the suture locking mechanism. As will be described further below, specific embodiments of tool 200 can easily disassemble when the suture locking mechanism is locked.

The illustrated embodiment of base 202 includes an inner surface 210, an outer surface 212, and a rest surface 214. A lateral boss or flange 216 extends outward from outer surface 212. Inner surface 210 in this embodiment has a concave curvature, and in particular embodiments has the same curvature as a suture locking mechanism with which tool 200 is to be used. For example, a part-circular curvature of inner surface 210 may provide a flush fit against a circular portion of a locking mechanism, e.g. a base such as body 124 of mechanism 120 described above. In other embodiments inner surface 210 may be partially curved, or have a configuration designed to accommodate or hold a suture locking mechanism.

Outer surface 212 has a convex curvature in the illustrated embodiment that is parallel to at least a portion of the concave curvature of the example of inner surface 211. Outer surface 212 extends on either side of lateral boss 216 in this embodiment. Outer surface 212 has a portion that is taller in height adjacent rest surface 214 than is the remainder of outer surface 212. Rest surface 214 is at least partially concave in this embodiment, and as illustrated may be a cylindrical concave surface. Rest surface 214 adjoins outer surface 211 and has the height of the taller portion of outer surface 212. Surface 214 accommodates a user's finger, thumb, holding tool, or other object as a way to hold or grip base 202. Rest surface 214 is at one end of base 202 in this embodiment, so that a holder on or against rest surface 214 provides the largest possible amount of leverage, torque or support for base 202.

Lateral boss 216 projects from outer surface 212, and provides another surface or element for a user to hold, support or push against in using tool 200. In the illustrated embodiment, boss 216 has an elliptical or torpedo shape, ending in a rounded point or line outboard of outer surface 212. Boss 216 has a portion that extends below outer surface 212, e.g. having a height the same as the taller portion of outer surface 212 and rest surface 214. The portion of boss 216 below surface 212 in the illustrated embodiment 216 has a thickness measured perpendicular to surface 212 that is less than that of the portion of boss 216 joining surface 212. An open area or gap 218 is thus defined in base 202 generally bordered by the lower portion of boss 216, the lower portion of outer surface 212, and an undersurface 220 parallel to an oversurface 222. A portion of a suture locking mechanism fits in such a gap, as will be discussed further below.

At the end opposite from rest surface 214 in this embodiment is a slot 226 that is parallel to surfaces 220 and 222. Slot 226 extends from the end of base 202 opposite rest surface 214 to a point adjacent boss 216 in the illustrated embodiment. Slot 226 is bounded by an upper surface 228 and a lower surface 230. Grooves 232, 234 are in respective surfaces 228, 230, and each include an entry portion 236 leading from an end surface, which in the illustrated embodiment is slightly curved, and a rounded (e.g. circular) end portion 238. Entry portion 236 is concave facing boss 216 and rest surface 214. Rounded portion 238 is off-center with respect to entry portion 236. For example, if portion 238 is circular, its center is not on a longitudinal axis of entry portion 236. Each portion 238 is off-center to the same side of each entry portion 236, so that they are aligned along an axis perpendicular to surfaces 228 and 230. An inner part of base 202 has a projection 239 in this embodiment, configured to connect with an opening in the side of a suture locking mechanism to be closed.

Bearing member 204 connects with and is rotatable with respect to base 202. Bearing member 204 has an outer wall 240 having a flat or slightly concave portion 242 that adjoins and curves into a slightly convex portion 244. An inner wall 246 has a smooth concave curvature that does not follow the curvature of outer wall 240. Inner wall 246 meets outer wall 240 in a curved end or point 248. Opposite end 248 is a tongue 250 configured to fit within slot 226 to form a pivotable joint. Tongue 250 is generally planar, having a rounded forward end 252, and is bounded by a planar rearward boss 254 that extends above and below tongue 250. Tongue 250 also includes upper and lower planar surfaces 256, 258, from which respective projections 260, 262 protrude. Projections 260, 262 are essentially identical in the illustrated embodiment, in the form of a rounded rectangle. Shorter ends 264 have a similar or identical convex curve. Longer sides 266, 268 are respectively concavely and convexly curved and parallel to each other and generally point toward forward end 252. The length of projections 260, 262 is approximately the same as a diameter of rounded portion 238 of grooves 232, 234 in this embodiment. For example, curved ends 264 may have the same curvature as rounded portions 238 of grooves 232, 234, so that projections 260, 262 can fit and rotate within rounded portions 238.

In use, bearing member 204 is fitted to base 202 by inserting tongue 250 into slot 226. To do so, projections 260, 262 are oriented so that they generally align with entry portions 236 of grooves 232, 234, and tongue 250 is then inserted so that projections 260, 262 travel along portions 236 until projections 260, 262 occupy round portions 238 of grooves 232, 234. In the illustrated embodiment, that insertion orientation has inner surface 210 of base 202 and inner surface 246 of bearing member 204 generally facing each other, e.g. in a relatively closed position (similar or identical to the orientation of tool 200 in FIG. 26). Once projections 260, 262 have been moved through straight portions 236 and into round portions 238, bearing member 204 can be pivoted with respect to base 202 (or base 202 and bearing member 204 are pivoted with respect to each other) to a relatively open position (e.g. FIG. 18 or 25), with inner surfaces 210 and 246 moving generally away from each other. Projections 260, 262 turn within round portions 238 of grooves 234 (i.e. a pivoting axis is at or near the center of rounded portions 238), as the upper and lower surfaces of tongue 250 slide along the upper and lower surfaces bounding slot 226. In particular embodiments, tool 200 can be opened by such pivoting to a maximal extent at which end surface 241 of bearing member 204 contacts boss 254 of base 202, with such contact preventing further pivoting in an opening direction, while pivoting to close tool 200 (moving one or both of base 202 and bearing member 204 relatively toward each other) is not inhibited. In that maximally opened position, in particular embodiments the inner surfaces 210 and 246 or respective portions of them form a part of the same circle.

Tool 200 may be provided in the assembled condition, as discussed above, so that a user may use it as soon as is needed. The following description will refer to closing an example of a suture locking mechanism 120 described above. It will be understood that tool 200 or other embodiments consistent with its principles could be used with other types of suture locking mechanisms.

Once mechanism 120 has been placed as desired by the physician or other user and is ready for closure and locking, i.e. a placed suture is through or otherwise engaged with it, tool 200 is engaged around mechanism 120. One advantage of this embodiment of tool 200 is that the user needs only one hand to operate it, so that his or her other hand can remain on mechanism 120 and/or the suture it is to hold to maintain necessary tension or positioning for them. Tool 200 in its open (e.g. maximally open) position is placed around mechanism 120, for example so that inner surface 246 of bearing member 204 is engaged with or adjacent to an outer portion of handle 158 of mechanism 120, and so that inner surface 210 of base 202 is engaged with or adjacent to an outer side 133 of body 124 of mechanism 120. For example, interior surfaces of boss 216 and/or a surface inside of portions 212, 214 of base 202 engage a side of body 124, and/or surface 220 of base 202 engages the top of body 124. Closure tool 200 when fit on or around mechanism 120 has the same axis of rotation (e.g. within rounded portions 238, as noted previously) as cam lever 128 with respect to body 124 (e.g. the axis through projections 186 and/or shaft 171). The user squeezes tool 200, e.g. with a thumb on rest surface 214 or contacting boss 216 of base 202 and a forefinger on outer concave surface 242 of bearing member 204, so that one or both of base 202 and bearing member 204 pivot toward a closed configuration of tool 200.

If not already so engaged, such squeezing engages base 202 and bearing 204 with respective parts of mechanism 20 as indicated above, and pushes handle 158 into body 124. Squeezing tool 200 continues until mechanism 20 is locked, e.g. when channel 167a snaps over projection 180 in mechanism 120. In the illustrated embodiment, tool 200 is designed to be used with one or more specific mechanisms such that when the mechanism is fully locked, base 202 and bearing member 204 of tool 200 are oriented such that projections 260, 262 are essentially aligned with entry portions 236 of grooves 234. With that advantage, further squeezing or pushing on the parts of tool 200 creates a fulcrum at 270, tending to pivot projections 260, 262 back through entry portions 236 to disengage base 202 and bearing member 204. Such disengagement allows the parts of tool 200 to fall away from or otherwise be easily removed from around the suture locking mechanism. In that case, there is no need to use a finger or another tool to open or pry away a part of tool 200 from the mechanism.

While the description above notes the attachment or connection of tool 200 to mechanism 120 by a surgeon or other user, it will be understood that one or more embodiments of a suture locking mechanism (e.g. mechanism 20, 120) can be packaged or otherwise provided with one or more embodiments of a closing tool (e.g. 200) prior to opening, preparation or other use by a user. A package or kit (with or without suture anchors or other medical devices with which a suture locking mechanism may be used) may include one or more individual suture locking mechanisms paired with respective closing tools, or multiple suture locking mechanisms may be provided with at least one closing tool that may be used multiple times. In a particular example, a suture locking mechanism is provided to a user already connected or engaged to a closing tool, with the suture locking mechanism in an open position. In such a configuration, the suture locking mechanism and closing tool may be held together, as by interaction between parts of the suture locking mechanism and the closing tool (e.g. snap-fit, interference fit, non-permanent or mild adhesives) or other connection. Such a connection can preferably be overcome by finger pressure by the user operating the closing tool as discussed above. The suture locking mechanism/closing tool combination may be pre-loaded on a suture, or the suture may be threaded through the suture locking mechanism (as previously described) during use. When locking is desired, pressure on the closing tool as discussed above locks the suture locking mechanism to secure the suture and allows the closing tool to be removed, as by automatic or easy disassembly of the base and bearing members of the closing tool.

While the disclosure has been illustrated and described in detail in the drawings and foregoing description, the same is to be considered as illustrative and not restrictive in character, it being understood that only particular embodiments have been shown and described and that all changes, equivalents, and modifications that come within the spirit of the inventions defined by the following claims are desired to be protected. It will be understood that structures or other features described with respect to one particular embodiment or item may be used in connection or along with other features, items or embodiments included herein. All publications, patents, and patent applications cited in this specification are herein incorporated by reference as if each individual publication, patent, or patent application were specifically and individually indicated to be incorporated by reference and set forth in its entirety herein.

What is claimed is:

1. A suture retention mechanism comprising:
a disk-shaped body having an upper surface, a lower surface, and an external side surface, and a channel defined through the side surface and parallel with the lower planar surface separating the body into upper and lower portions, the channel bounded in part by an upper face in the upper portion, a lower face in the lower portion, and one or more interior surfaces extending from the upper face to the lower face, the body further including a suture aperture, the suture aperture having respective openings in the upper surface, upper face, lower face and lower surface to define a continuous straight path from the upper surface to the lower surface and through the channel;
a cam lever positioned in and pivotally attached by an axle to the body to allow the cam lever to pivot in the channel between an open position and a closed position, the cam lever including a handle portion joined to an arm portion having an at least partially planar camming surface, so that the camming surface faces and moves along and past at least a portion of the interior surface of said body the cam is pivoted from the open position to the closed position, and wherein the axle forms an axis of rotation that is parallel to and offset from the suture aperture and is between the suture aperture and the external side surface, thereby intersecting the upper and lower faces;
wherein when the cam lever is in the open position, the suture aperture is at least partially unobstructed to allow a suture located in the suture opening to move longitudinally within the suture opening; and,
wherein when the cam is in the closed position, the cam obstructs the suture aperture so that a suture cannot move longitudinally within the suture opening.

2. The mechanism of claim 1, wherein a portion of the interior surface is curved, and the suture aperture is directly next to the curved portion of the interior surface.

3. The mechanism of claim 1, wherein a portion of the interior surface is flared, and a first portion of the handle faces the flared portion and an opposite portion of the handle is flush with or inside of the exterior side surface in the closed position.

4. The mechanism of claim 3, wherein in the closed position a gap exists between a tip of the handle and the body for access by a user to the handle.

5. The mechanism of claim 3, wherein the handle includes a length and opposing upper and lower surfaces and a tip, the tip having a diameter less than a width of the channel, wherein the distance between the upper and lower surfaces of the handle is not uniform along its length, so that at least one gap is formed between at least one of the upper and lower surfaces of the handle and at least one of the upper and lower faces of the body.

6. The mechanism of claim 1, further comprising at least one detent along the exterior side surface and extending from one of the upper and lower faces and into the channel.

7. The mechanism of claim 6, comprising first and second detents along the exterior side surface, the first and second detents having a first height at the exterior side surface and respective portions sloping from the exterior side surface to one of the faces bounding the channel, the first and second detents being directly opposite each other across the channel.

8. The mechanism of claim 1, wherein the interior surface includes a flat stop portion, wherein a portion of the cam lever engages the flat stop portion when the cam lever is in a maximally-open position.

9. The mechanism of claim 8, wherein when a portion of the cam lever engages the flat stop portion, an end portion of the arm of the cam lever contacts a curved portion of the interior surface.

10. The mechanism of claim 1, wherein the axle includes a pin that extends through a portion of the cam lever and engages the body through openings in the upper and lower face of the body.

11. The mechanism of claim 10, wherein the pin is fixed with respect to one of the body and the cam lever.

12. The mechanism of claim 10, wherein the body includes a pin aperture with respective openings in at least one of the upper surface and the lower surface of the body and in the upper and lower faces of the body for holding the pin.

13. The mechanism of claim 1, wherein a portion of said cam protrudes from said body when said cam is in said open position.

14. The mechanism of claim 1, wherein the opening in the lower surface of the body of the suture aperture is in the center of the lower surface of the body.

15. The mechanism of claim 1, wherein when the cam lever is in said closed position, the cam lever covers said suture opening, and an outside edge of the cam lever is approximately flush with the exterior side surface of the body.

16. A suture retention mechanism comprising:
a body including a suture aperture, the suture aperture having a longitudinal axis and respective openings in an upper surface and a lower surface to define a continuous straight path from the upper surface to the lower surface;
a cam lever positioned in and pivotally attached by an axle to the body to allow the cam lever to pivot into and out of the body between an open position and a closed position, the cam lever including an arm portion with a side camming surface, wherein the axle forms an axis of rotation that is parallel to and offset from the longitudinal axis of the suture aperture and is between the suture aperture and an external side surface of the body, wherein when the cam lever is pivoted around the axis of rotation toward the closed position the camming surface moves laterally across the suture aperture; and
wherein when the cam lever is in the open position, the suture aperture is at least partially unobstructed to allow a suture located in the suture opening to move longitudinally within the suture opening; and,
wherein when the cam lever is in the closed position, the cam lever obstructs the suture aperture so that a suture cannot move longitudinally within the suture opening.

17. The mechanism of claim 16, wherein the body includes a channel into and out of which the cam lever is pivotable, the channel being partially bounded by a plurality of interconnected internal surfaces, wherein one of the internal surfaces is a stop surface that engages the cam lever at a maximally-open position to prevent further outward pivoting of the cam lever, wherein one of the internal surfaces is a curved surface adjacent the suture aperture and facing the camming surface of the cam lever, and wherein one of the internal surfaces is a flared surface defining an area for accommodating a handle portion of the cam lever when the cam lever is in the closed position.

18. The mechanism of claim 16, wherein the body includes at least one detent for holding the cam lever in the closed position.

19. The mechanism of claim 16, wherein the cam lever includes a curved handle portion that extends outside of the external side surface of the body when the cam lever is in the open position, and is flush with or inside of the external side surface of the body when the cam lever is in the closed position.

20. The mechanism of claim 19, wherein when the cam lever is in the closed position, at least one gap exists between the handle portion and the body to assist a user in accessing the handle portion to open the cam lever.

21. The mechanism of claim 16, wherein the cam lever includes a ridge and the body has a projection, and wherein the ridge snaps over the projection as the cam lever goes to the closed position.

22. A closure mechanism usable to close a suture locking mechanism, comprising:
a base having an inner surface for engaging a first part of the suture locking mechanism, a concave surface at a first end and a slot at a second end, the slot defined at least in part by upper and lower surfaces, each having a respective groove, with each respective groove having a curved entry portion and a rounded end portion;
a bearing member having an inner surface for engaging a second part of the suture locking mechanism, the bearing member having a concave surface at a first end and a tongue at a second end, the tongue having an upper and lower surface, each of which has a respective projections having convex end surfaces, one convex side surface and one concave side surface,
wherein the respective projections are located within and pivotable around a pivot axis within the respective rounded end portions of the respective grooves,
and wherein when the closure mechanism engages the suture locking mechanism to close the suture locking mechanism the pivot axis is aligned with an axis about which the second part of the suture locking mechanism rotates with respect to the first part.

23. The closure mechanism of claim 22, wherein when the suture locking mechanism is closed sufficiently to lock a suture, the respective projections are slidable through the entry portion of the respective grooves so that the base and bearing member can be disassembled.

24. The closure mechanism of claim 22, wherein the base and bearing member are pre-assembled and engaged to the suture locking mechanism prior to receipt and use by a user.
